(19) United States
(12) Patent Application Publication
(10) Pub. No.: US 2023/0321085 A1
(43) Pub. Date: Oct. 12, 2023

(54) COMPOUND FOR USE IN THE TREATMENT OF DRY MOUTH

(71) Applicant: ASAHI PHARMA CO., LTD., Ibaraki (JP)

(72) Inventors: Michiel Henricus De Vries, EC Weep (NL); Dorien Groenendaal, AT Hoofddorp (NL)

(73) Assignee: ASAHI PHARMA CO., LTD., Ibaraki (JP)

(21) Appl. No.: 18/044,270

(22) PCT Filed: Sep. 13, 2021
(86) PCT No.: PCT/JP2021/034445

§ 371 Date: Mar. 7, 2023

(30) Foreign Application Priority Data
Sep. 11, 2020 (EP) 20195704.0

(51) Int. Cl. A61K 31/497; A61K 45/06; A61K 31/4178; A61P 1/02

(52) U.S. Cl. CPC A61K 31/497; A61K 45/06; A61K 31/4178; A61P 1/02

(57) ABSTRACT

The present invention is concerned with the use of compound

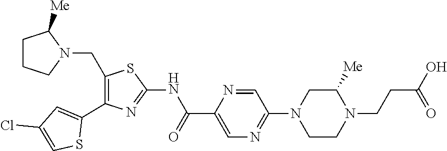 [SMILES: C[C@@H]1CCCN1Cc1sc(NC(=O)c2cnc(N3CCN(CCC(=O)O)[C@@H](C)C3)cn2)nc1-c1cc(Cl)cs1]

in the treatment of dry mouth.

### CROSS-REFERENCED APPLICATIONS

This is a National Phase Application of International Application No. PCT/JP2021/034445, filed on Sep. 13, 2021, which claimed priority to EP Application No. 20195704.0, filed on Sep. 11, 2020, both of which are incorporated herein in their entireties by reference thereto.

### TECHNICAL FIELD

The present invention relates to a compound for use in the treatment of dry mouth.

### BACKGROUND ART

Dry mouth and oral dryness are general terms that encompass two medical entities: xerostomia and hyposalivation. Xerostomia is the subjective complaint of oral dryness and is medically classified as a symptom. Hyposalivation is the objective reduction in salivary secretion, as the consequence of reduced salivary gland function. Generally, these conditions are closely related to each other: a reduced salivary flow is the most frequent cause of xerostomia, and it is generally accepted that hyposalivation causes the symptom of oral dryness when salivary secretion rate is reduced by at least 50% (https://www.eaom.eu/pdf/content/dry_mouth.pdf). Dry mouth can also cause other symptoms, such as rough tongue, mouth sores, and cracked lips. It may also lead to difficulties in swallowing.

Dry mouth can have many causes. It often results from dehydration. Radiation therapy for head and neck cancer often damages salivary glands and causes hyposalivation. Some diseases, such as diabetes and Sjogren syndrome, can also affect saliva production. More seriously, dry mouth can be a side effect of certain medications. In fact, there are more than 1,800 commonly prescribed medications that list dry mouth as a side effect.

Dry mouth is especially noteworthy as a side effect of anticholinergic drugs. Salivary secretion is under the control of autonomic nervous activity, the rate of fluid output being regulated by parasympathetic activity mediated by muscarinic acetylcholine receptors (mAChRs) in the salivary gland cells responsible for saliva secretion (Baum, Ann NY Acad Sci. 1993; 694:17-23; Cook et al., Physiology of the Gastrointestinal Tract. Raven, New York: 1994. pp. 1061-1117). In terms of mAChR subtype, both the M1 and M3 subtypes have been reported to be present in salivary glands, however, results have not been conclusive. While it is still under discussion which mAChR subtypes contribute to cholinergic control of salivation and to what extent, especially under physiological conditions, it is observed that, antagonists of the M3 muscarinic receptor often result in dry mouth as a side effect.

WO 07/027675 discloses pharmaceutical compositions comprising a therapeutically effective amount of a first compound and a therapeutically effective amount of a second compound. The first compound is an antimuscarinic or an anticholinergic agent and the second compound causes stimulation of salivary glands. The second compound of WO 07/027675 is a cholinergic agonist inter alia selected from the group consisting of pilocarpine, cevimeline, and amifostine or a pharmaceutically acceptable salt or prodrug thereof. The combination is based on the concept that at the receptor level, the adverse effect of the M2/M3 muscarinic antagonists is counteracted or negated with cholinergic agents that work in the opposite direction, but in concert with the intended therapy. In a case study, WO 07/027675 describes the combined use of oxybutynin and pilocarpine, whereby a combination of 5 mg oxybutynin and 5 mg pilocarpine results in a slightly increased saliva production compared to baseline.

WO 19/189766 discloses biarylamide compounds as M3 positive allosteric modulators and their pharmaceutical uses including dry mouth. The document explains that a positive allosteric modulator binds to an allosteric site other than the ligand binding site and mainly causes structural changes in the receptor to increase the binding force between the agonist and the receptor and change the agonist signal level. The positive allosteric modulators are said not to show agonistic effects on their own in vivo but enhance the activities of M3 agonists.

Pilocarpine shows dose-dependent effects on salivation. In human trials, it was observed that 2.5, 5 and 10 mg pilocarpine increased salivation (see Example 1 below). Yet, at clinically relevant doses (5 mg and 10 mg qd), a significant number of the patients suffered from sweating. Pilocarpine therefore alleviates the dry mouth symptoms, but it leads to hyperhidrosis as a further side effect. Moreover, experiments in rats have shown that the effect of pilocarpine on salivation is only short-lived and that initial salivation levels are approached after about 60 minutes (FIG. 1).

### SUMMARY OF INVENTION

Accordingly, there is further need for medication that alleviates dry mouth without causing further side effects such as hyperhidrosis. It would also be beneficial to have a medication with a long-lasting effect.

The present inventors have found that this need can be met with the compound of the invention. The finding underlying the present invention is surprising in at least three aspects. First, the compound according to the present invention was found to be useful in the treatment of dry mouth in the absence of treatment with either a muscarinic agonist or an anticholinergic agonist. Second, it was found that the compound for use according to the present invention is useful for the treatment of dry mouth without causing sweating. Third, the effect of the compound of the invention is long-lasting.

The compound for use in the present invention and methods of making the same are known from EP-A 3 153 511. EP-A 3 153 511 describes the compound for use according to the present invention and its use for treating bladder and urinary tract diseases associated with bladder contractions via a muscarinic M3 receptor. EP-A-3 153 511 does not disclose or suggest that the compounds disclosed therein may be useful in the treatment of dry mouth.

The compound for use according to the present invention is the compound of Example 144 in EP-B 3 153 511 in its free form or its esters or pharmaceutical salts thereof such as the di-maleate:

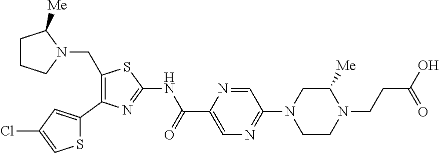 [SMILES: C[C@@H]1CCCN1Cc1sc(NC(=O)c2cnc(N3CCN(CCC(=O)O)[C@@H](C)C3)cn2)nc1-c1cc(Cl)cs1]

### BRIEF DESCRIPTION OF DRAWINGS

FIG. 1 shows the effect of pilocarpine (oral) on salivation in awake rats.

FIG. 2 shows the effect of the compound of the invention (oral) in awake rats.

FIG. 3 shows the results of a clinical study with pilocarpine.

FIG. 4 shows the results of a clinical study with a single dose of the compound of the invention.

FIG. 5 shows the results of a clinical study with multiple doses of the compound of the invention.

FIG. 6 show the results of a clinical study with a reference M3 PAM.

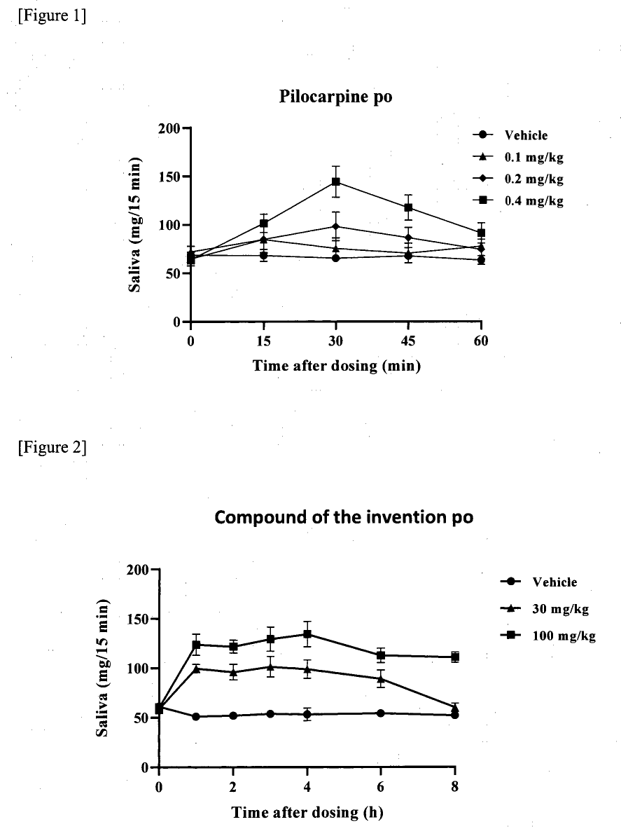

### DESCRIPTION OF EMBODIMENTS

In addition, a pharmaceutically acceptable prodrug of the compound of the invention is also described. The pharmaceutically acceptable prodrug refers to a compound having a group which can be converted into an amino group, a hydroxyl group, a carboxyl group, or the like, by solvolysis or under a physiological condition. Examples of the groups forming the prodrug include those as described in Prog. Med., 5, 2157-2161 (1985) or “Pharmaceutical Research and Development” (Hirokawa Publishing Company, 1990), vol. 7, Drug Design, 163-198.

Moreover, the salt of the compound is a pharmaceutically acceptable salt of the compound, and the compound may form an acid addition salt or a salt with a base, depending on the kinds of the substituents in some cases. Specifically, examples thereof include acid addition salts with inorganic acids such as hydrochloric acid, hydrobromic acid, hydroiodic acid, sulfuric acid, nitric acid, and phosphoric acid, and with organic acids such as formic acid, acetic acid, propanoic acid, oxalic acid, malonic acid, succinic acid, fumaric acid, maleic acid, lactic acid, malic acid, mandelic acid, tartaric acid, dibenzoyl tartaric acid, ditolyl tartaric acid, citric acid, methanesulfonic acid, ethanesulfonic acid, benzenesulfonic acid, p-toluenesulfonic acid, aspartic acid, and glutamic acid, and salts with metal anions such as sodium, potassium, magnesium, calcium, and aluminum, and with organic bases such as methylamine, ethylamine, and ethanolamine, salts with various amino acids such as acetyl leucine, lysine, and omithine, or derivatives of amino acids, ammonium salts, and others.

In addition, the present invention also includes various hydrates or solvates, and crystal polymorph substances of the compound and a salt thereof. In addition, the present invention also includes compounds labelled with various radioactive or non-radioactive isotopes.

A pharmaceutical composition including the compound as an active ingredient can be prepared using an excipient which is commonly used in the art, that is, an excipient for a pharmaceutical preparation, a carrier for a pharmaceutical preparation, and the like, according to a method usually used.

Administration can be accomplished either by oral administration via tablets, pills, capsules, granules, powders, solutions, and the like, or parenteral administration via injections, such as intraarticular, intravenous, and intramuscular injections, suppositories, transdermal liquid preparations, ointments, transdermal patches, transmucosal liquid preparations, transmucosal patches, inhalers, and the like. Oral administration is preferred.

As a solid composition for oral administration, tablets, powders, granules, and the like are used. In such a solid composition, one kind or two or more kinds of the active ingredients are mixed with at least one inactive excipient. In a conventional method, the composition may contain inactive additives such as a lubricant, a disintegrating agent, a stabilizer, or a solubilization assisting agent. If necessary, tablets or pills may be coated with a sugar or with a film of a gastric or enteric coating substance. Tablets are the preferred form of administration.

The liquid composition for oral administration includes pharmaceutically acceptable emulsions, solutions, suspensions, syrups, elixirs, or the like, and includes generally used inert diluents, for example, purified water or ethanol. The liquid composition may also include auxiliary agents such as a solubilization assisting agent, a moistening agent, and a suspending agent, sweeteners, flavors, aromatics, and antiseptics, in addition to the inert diluent.

The injections for parenteral administration include sterile aqueous or non-aqueous solution preparations, suspensions, or emulsions. The aqueous solvent includes, for example, distilled water for injection and saline. Examples of the non-aqueous solvent include alcohols such as ethanol. Such a composition may further include a tonicity agent, an antiseptic, a moistening agent, an emulsifying agent, a dispersing agent, a stabilizing agent, or a solubilizing assisting agent. These are sterilized, for example, by filtration through a bacteria retaining filter, blending of a bactericide, or irradiation. In addition, these can also be used by preparing a sterile solid composition and dissolving or suspending it in sterile water or a sterile solvent for injection prior to its use.

Examples of the agent for external use include ointments, hard plasters, creams, jellies, cataplasms, sprays, and lotions. The agent further contains generally used ointment bases, lotion bases, aqueous or non-aqueous liquid preparations, suspensions, emulsions, or the like.

As transmucosal delivery is contemplated, e.g. by inhalation or transnasal delivery, the pharmaceutical composition can take the form of a solid, liquid, or semi-solid and it can be prepared in accordance with any method known in the art. For example, a known excipient, and also a pH adjusting agent, an antiseptic, a surfactant, a lubricant, a stabilizing agent, a thickening agent, or the like may be appropriately added thereto. For the administration, an appropriate device for inhalation or blowing can be used. For example, a compound may be administered alone or as a powder of formulated mixture, or as a solution or suspension in combination with a pharmaceutically acceptable carrier, using a known device or sprayer such as a metered administration inhalation device. A dry powder inhaler or the like may be for single or multiple administration use, and a dry powder or a powder-containing capsule may be used. Alternatively, this may be in a form such as a pressurized aerosol spray that uses an appropriate propellant agent, for example, a suitable gas such as chlorofluoroalkanes, and carbon dioxide, or other forms.

In the case of oral administration, the daily oral and transmucosal dose is usually from 0.001 mg/kg body weight to 100 mg/kg body weight, preferably from 0.01 mg/kg body weight to 30 mg/kg body weight, more preferably from 0.01 mg/kg body weight to 10 mg/kg body weight and most preferably 0.01 mg/kg body weight to 5 mg/kg body weight. The daily oral or transmucosal dose can be administered in one portion or divided into 2 to 4 portions. In the case of intravenous administration, the daily dose corresponds to the above oral doses, but it is preferably lower by a factor of 10. The dose is appropriately decided in response to the individual case by taking the symptoms, the age, and the gender, and the like into consideration. Oral or transmucosal daily doses of 30 mg or higher especially between 50 and 200 mg such as 100 to 150 mg are preferential.

Although there are differences depending on the route of administration, the dosage form, the administration site, and the type of the excipient or additive, the pharmaceutical composition of the present invention comprises 0.01% by weight to 100% by weight or preferably 0.01% by weight to 50% by weight of the compound or a salt or ester thereof as the active ingredient.

The compound may be used in combination with various agents that cause dry mouth, such as anticholinergic agents. Examples for anticholinergic agents are atropine, aclidinium, benztropine, cyclopentolate, diphenhydramine, doxylamine, dimenhydrinate, diclomine, darifenacin, flavoxate, hydroxyzine, ipatropium, mebeverine, oxybutynin, pirenzepine, procyclidine, scopolamine, solifenacin, topicamide, tolterodine, tiotropium, trospium and their pharmaceutically acceptable salts.

The compound may also be administered in combination with parasympathomimetica such as pilocarpine. Parasympathomimetic drugs may be used to reduce dry mouth, yet they cause hyperhidrosis. A combination of such parasympathomimetic compounds with the compound according to the invention allows to reduce the dose of the parasympathomimetic and thereby treat dry mouth while reducing or eliminating the unwanted sweating.

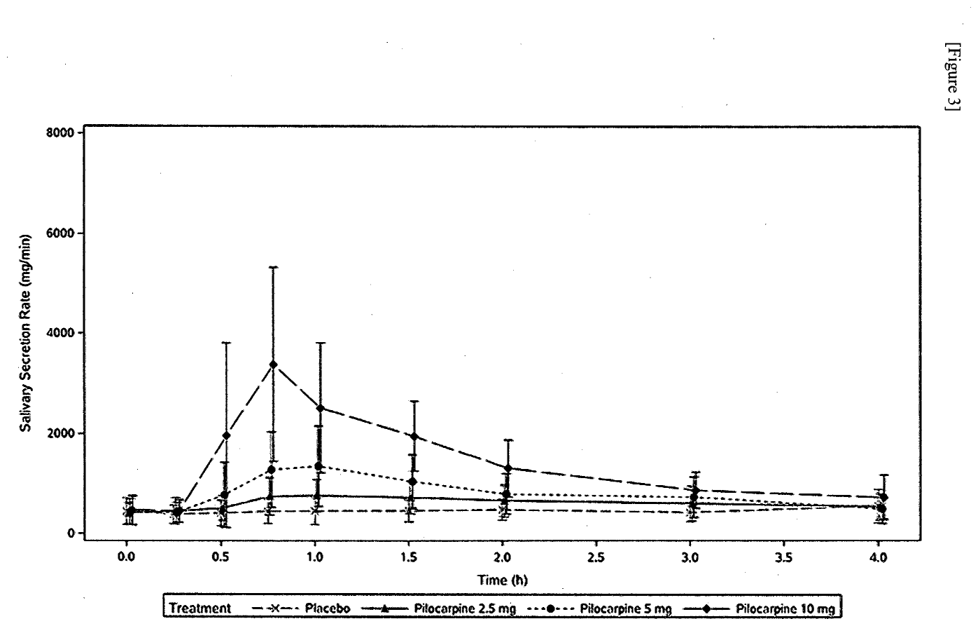

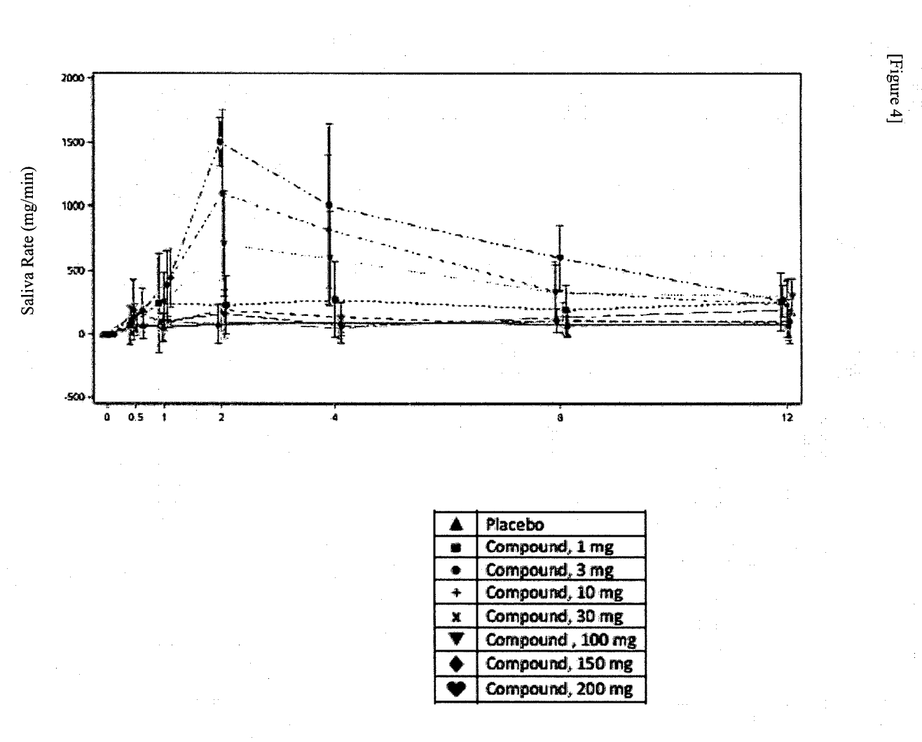

Surprisingly, the compound of the invention can treat dry mouth when used on its own. It also does not cause hyperhidrosis. Moreover, experiments have shown that its effect on salivation is long-lasting (FIG. 2 as well as FIGS. 3, 4 and 5). Specifically, while pilocarpine only showed a short-term effect in the rat experiment (approximately 60 min or less), the compound of the invention had an effect lasting for 8 hours or more in the same experiment (oral administration of the active to awake rats).

The compound of the invention and the other therapeutic agents may be administered simultaneously, or separately and continuously, or at a desired time interval. The preparations to be co-administered may be a blend or may be prepared individually.

### EXAMPLES

### Example 1 (Reference

A Phase 1 study to investigate the effect of pilocarpine on salivary secretion was conducted in 12 healthy subjects. The study was conducted as a randomized, subject- and investigator-blinded, placebo-controlled, oral dose clinical study using a 5-way balanced extra period design.

TABLE 1
Dosing Plan for Each Treatment Sequence

| | Period 1 | Period 2 | Period 3 | Period 4 | Period 5 |
|---|---|---|---|---|---|
| Sequence 1 | 2.5 mg | 5 mg | 10 mg | Placebo | Placebo |
| Sequence 2 | 5 mg | Placebo | 2.5 mg | 10 mg | 10 mg |
| Sequence 3 | 10 mg | 2.5 mg | Placebo | 5 mg | 5 mg |
| Sequence 4 | Placebo | 10 mg | 5 mg | 2.5 mg | 2.5 mg |

Pilocarpine capsules containing 2.5 mg were administered orally (1 capsule for a 2.5 mg dose, 2 capsules for a dose of 5.0 mg pilocarpine and 4 capsules for a dose of 10 mg pilocarpine). Subjects were admitted to the clinical unit on day 1 and were residential for a period of 6 days and 5 nights. Subjects were randomized to 1 of 4 treatment sequences on day 1 prior to study drug administration. Each study period was approximately 1 day and subjects received the different oral doses of the study drug (pilocarpine or placebo to match) in periods 1 through 5. There was a 4-hour saliva collection period following every dose and a washout period of at least 24 hours between doses.

To assess the pharmacodynamic effect of pilocarpine on saliva secretion, saliva samples were collected over 5-minute intervals from pre-dose until 4 hours post-dose at the timepoints 0.5, 0.75, 1.0, 1.5, 2.0, 3.0 and 4.0 h. The salivary secretion rate over time is shown in FIG. 3.

The most commonly reported side effect for periods 1 to 4 was hyperhidrosis. At a pilocarpine dose of 5 mg, this side effect was reported by one participant (8.3%). At a pilocarpine dose of 10 mg, this side effect was reported by 50% of the participants.

### Example 2

In the first part of a randomized, subject- and investigator-blinded, placebo-controlled, single ascending oral dose, parallel group study comprising of 7 cohorts (cohorts 1.1 to 1.7) the compound of the invention was administered at dose levels of 1, 3, 10, 30, 100, and 150 mg. Each cohort consisted of 8 healthy non-elderly male and female subjects who were randomized to receive the compound or matching placebo in a 3:1 ratio.

After a screening period of 21 days, eligible subjects who had signed an informed consent form (ICF) to participate in the study were residential for a single treatment period of 5 days/4 nights. Subjects were admitted to the clinical unit on day −1. After randomization on day 1, subjects received a single oral dose of the compound or matching placebo under fasting. Each cohort was completed with an end-of-study visit, which took place 5 to 9 days after the last pharmacokinetic sample had been collected (or early discontinuation from the study).

The second part of the study investigated, multiple ascending oral doses in 4 cohorts (cohorts 2.1 to 2.4). Each cohort consisted of 12 healthy non-elderly male and female subjects, one optional cohort (cohort 2.5) consisting of 12 healthy nonelderly male and female subjects and one cohort (cohort 2.6) consisting of 12 healthy elderly male and female subjects who were randomized to receive the compound or matching placebo in a 3:1 ratio.

Actual dose levels were 10, 30, 100 and 150 mg of the compound (cohorts 2.1 to 2.4, respectively). The optional cohort 2.5 was not enrolled. The actual dose level for the elderly cohort (cohort 2.6) was 150 mg of the compound.

Sentinel dosing was employed for all cohorts. The first 2 subjects within a cohort (1 subject received the compound and 1 subject received matching placebo) were dosed at least 5 minutes apart. These 2 subjects were monitored for safety for 48 hours after receiving the first dose while dosing was continued. The remaining 10 subjects (8 with the compound and 2 with matching placebo) were divided into 2 sub-cohorts and were dosed at least 48 hours after the first 2 subjects with 48 hours between the 2 sub-cohorts.

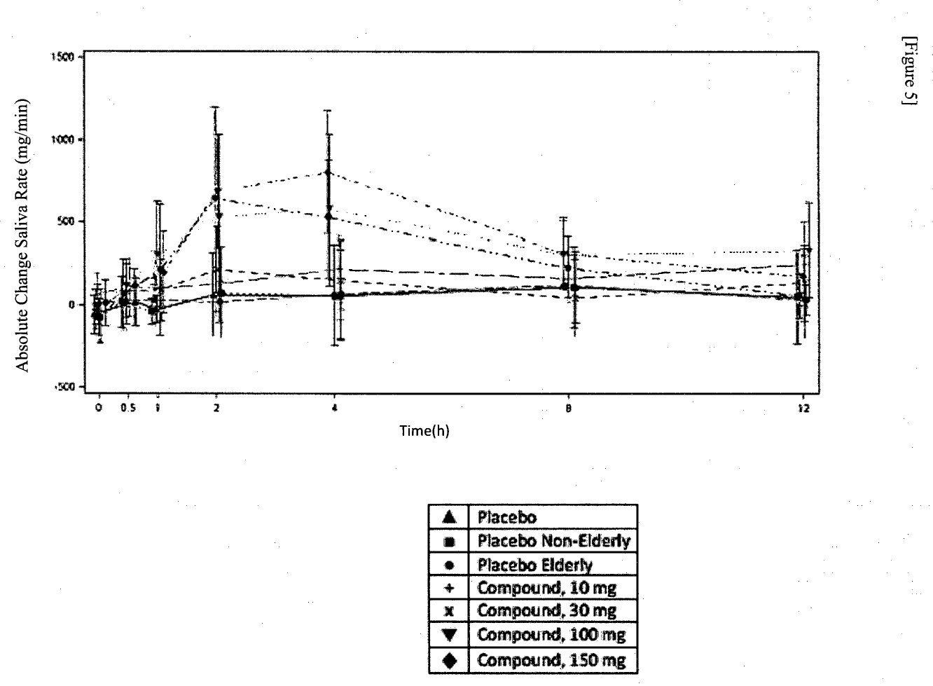

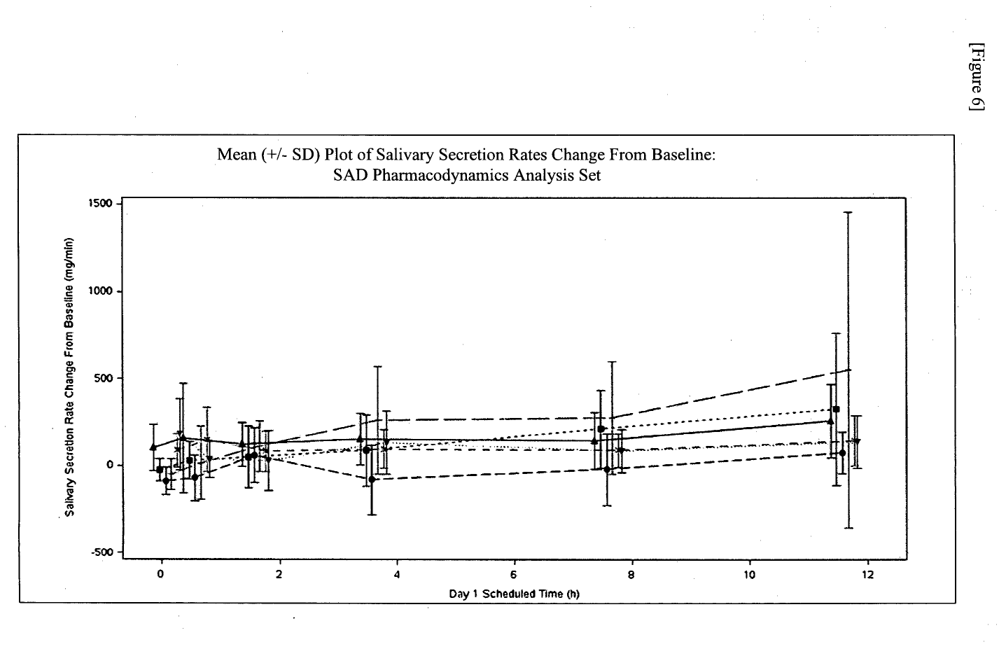

In both studies the compound increased salivary secretion rates, while hyperhidrosis was not reported as a side effect. The results of the first study (single dose) are shown in FIG. 4 and the results of the second study (multiple dose) are shown in FIG. 5.

### Example 3 (Reference

In a study similar to the first study in Example 2, a known M3 positive allosteric modulator reference compound (example 8 of EP 2 963 036) with the following structure

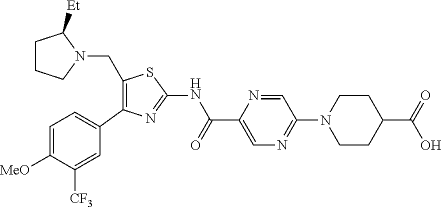 [SMILES: CC[C@@H]1CCCN1Cc1sc(NC(=O)c2cnc(N3CCC(C(=O)O)CC3)cn2)nc1-c1ccc(OC)c(C(F)(F)F)c1]

was administered in dose levels of 1, 3, 10, 30 and 90 mg. The results are shown in FIG. 6. The data show that other M3 positive allosteric modulators alone do not significantly impact salivation.

The above data indicates that the compound of the present invention is beneficial in that it increases salivation on its own. Animal experiments indicate that the effect is long-lasting. Moreover, clinical studies do not suggest that the compound of the invention causes hyperhidrosis. Hence the compound of the invention seems suited to remove the most serious side effect of the current dry mouth treatments.

What is claimed is:

1. A method for treating dry mouth by administering a compound with the following formula 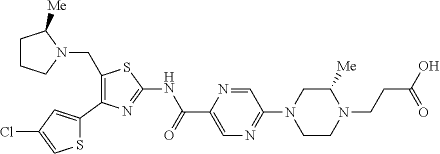 [SMILES: C[C@@H]1CCCN1Cc1sc(NC(=O)c2cnc(N3CCN(CCC(=O)O)[C@@H](C)C3)cn2)nc1-c1cc(Cl)cs1]
or pharmaceutically acceptable salts or esters thereof, to a subject who needs the treatment of dry mouth.

2. The method according to claim 1, wherein the compound is administered as a maleic acid salt, especially the dimaleate.

3. The method according to claim 1, wherein the dry mouth is associated with Sjörgren's disease, radiation or drug therapy, such as therapy with anticholinergic antagonists.

4. The method according to claim 1, wherein the compound is co-administered with an anticholinergic antagonist.

5. The method according to claim 4, wherein the anticholinergic antagonist is selected from atropine, aclidinium, benztropine, cyclopentolate, diphenhydramine, doxylamine, dimenhydrinate, diclomine, darifenacin, flavoxate, hydroxyzine, ipatropium, mebeverine, oxybutynin, pirenzepine, procyclidine, scopolamine, solifenacin, topicamide, tolterodine, tiotropium, trospium and their pharmaceutically acceptable salts.

6. The method according to claim 1, wherein the compound is administered in combination with pilocarpine.
